(19) United States
(12) Patent Application Publication
(10) Pub. No.: US 2020/0129176 A1
(43) Pub. Date: Apr. 30, 2020

(54) SUTURE PACKAGING

(71) Applicant: Peters Surgical, Bobigny Cedex (FR)

(72) Inventors: Thomas Halfon, Gournay Sur Marne (FR); Caroline Camedda, Groslay (FR); Laurent Kirchoffer, Bobigny (FR)

(73) Assignee: Peters Surgical, Bobigny Cedex (FR)

(21) Appl. No.: 16/499,493

(22) PCT Filed: Mar. 30, 2018
(86) PCT No.: PCT/EP2018/058359

§ 371 Date: Sep. 30, 2019

(30) Foreign Application Priority Data
Mar. 30, 2017 (FR) 1752700

(51) Int. Cl. A61B 17/06; A61B 50/00

(52) U.S. Cl. CPC A61B 17/06114; A61B 17/06133; A61B 2050/0084; A61B 2017/06142; A61B 50/00

(57) ABSTRACT

Packaging for a suture, comprising:
a base (1) comprising a receiving zone (6, 8) for receiving a suture,
a lid (62),
means for locking the lid (62) to the base (1), comprising a first slot (58) formed in one among the base (1) and the lid (62), and a first flap (72) formed in the other among the base (1) and the lid (62),
   wherein the first flap (72) has a shape and dimensions adapted to:
be engaged through the first slot (58) when the lid (62) has a first orientation with respect to the base (1), then
be blocked athwart the first slot (58) by rotation of the lid (62) from the first orientation to a second orientation of the lid with respect to the base in which the lid covers at least in part the suture receiving zone (6, 8).

### FIELD OF THE INVENTION

The invention relates to the field of packing surgical supplies.

The present invention relates more particularly to packaging for a suture.

### PRIOR ART

Known from the prior art is packaging for a suture in two parts, comprising a base and a lid.

The base comprises a receiving zone for receiving a suture. The packaging also comprises means for locking the lid to the base so as to form a closed package.

Various means for locking the lid to the base have been proposed.

In a first type of packaging for a suture, the lid is glued to the base as a result of a fusion process.

One disadvantage of such gluing is the use of chemical products and solvents which can migrate into the suture. Another disadvantage is that one or more flat zones are needed on the base that are sufficiently large for its contacting with the lid.

In a second type of packaging for a suture, the base has a peripheral wall, and a plurality of pins protruding from an inner surface of the peripheral wall toward the center of the base. The lid is made of a cardboard material having an outer perimeter substantially complementary to the inner surface of the peripheral wall. To close the packaging industrially, the lid is placed above the base, so that the edge of the lid is aligned with the inner surface of the peripheral wall. A tool equipped with teeth presses the lid against the pins, so that the lid is slightly deformed to pass over the pins and be clamped below the latter. Thus, the lid is held attached to the base at its outer edge, by means of the plurality of pins.

However, the positioning of the lid of such packaging industrially is difficult to implement; a large number of pins is in fact needed so that the lid can be solidly attached to the base, which makes the shape of the base, and hence its manufacture, more complex. Moreover, the edge of the lid must be perfectly aligned with the peripheral wall of the base, failing which the tool equipped with teeth presses too strongly on the cardboard lid and consequently leaves ugly marks on it, or presses too strongly on the suture itself, at the risk of damaging it.

In a third type of packaging for a suture, several slots are formed in the base, and the lid comprises a plurality of rectangular flaps. To attach the lid to the base, each rectangular flap is engaged in a corresponding slot.

However, the flaps are likely to slip out of the slots and thus cause accidental disassembly of the lid and the packaging.

### DISCLOSURE OF THE INVENTION

One goal of the invention is to close packaging for a suture using locking means which overcome the aforementioned disadvantages of the prior art.

It is therefore proposed, according to a first aspect of the invention, packaging for a suture comprising:
a base comprising a receiving zone for receiving a suture,
a lid,
means for locking the lid to the base, comprising a first slot formed in one among the base and the lid, and a first flap formed in the other among the base and the lid,
   in which the first flap has a shape and dimensions adapted to:
be engaged through the first slot when the lid has a first orientation with respect to the base, then
be blocked athwart the first slot by rotation of the lid from the first orientation to a second orientation of the lid with respect to the base in which the lid covers at least in part the suture receiving zone.

Moreover, the first flap has a bent portion forming an abutment for retaining the first flap athwart the first slot when the lid is in the second orientation with respect to the base, and/or the lid has a flat surface intended to rest against the base after engagement of the first flap in the first slot, and in which the axis of rotation of the first orientation toward the second orientation is perpendicular to the flat surface.

The packaging can also comprise the following features, taken alone or in combination when that is technically possible.

The first flap can comprise a portion having:
a dimension, measured parallel to the first slot when the lid is in the first orientation with respect to the base, which is less than a length of the first slot, and
a dimension, measured parallel to the first slot when the lid is in the second orientation with respect to the base, which is greater than the length of the first slot.

The first flap has for example an L shape.

The first slot is preferably formed in the base, and the first flap formed in the lid.

The lid can comprise a sheet, made of cardboard for example, the first flap being formed by cutting out the sheet.

The packaging can comprise a boss protruding from the base, the boss having an apex intended to serve as a support for the lid, and a lateral wall connecting the apex to the base, the first slot being formed in the lateral wall of the boss.

The locking means can also comprise:
a second slot formed in one among the base and the lid,
a second flap formed in the other among the base and the lid, the second flap having a shape and dimensions adapted to be engaged in a second slot after engagement of the first flap through the first slot, then rotation of the lid from the first orientation to the second orientation with respect to the base, so as to block the lid in rotation with respect to the base in the second orientation.

The second slot can be formed in the base and the second flap be formed in the lid.

The two flaps can extend in two opposite directions. Alternatively, the two flaps extend so as to be able to be inserted respectively into the two slots in the same direction.

The base can comprise a peripheral wall having an inner surface, the lid can have an outer edge with a shape complementary to the inner surface of the peripheral wall, the peripheral wall being arranged so as to surround the lid when the lid has the second orientation after engagement of the first flap through the first slot.

The base can comprise a channel for retaining a suture in a wound position, and a parking zone for one end of the suture, the parking zone being surrounded by the retaining channel, and in which the first slot or the first flap is formed in the parking zone.

The packaging can comprise a suture received in the receiving zone of the base.

Also proposed, according to a second aspect of the invention, is a method for locking a lid of the packaging for a suture to a base of the packaging, the base comprising a receiving zone for receiving the suture, a first slot being formed in one among the base and the lid and a first flap being formed in the other among the base and the lid, the method comprising steps of:
engagement of the first flap through the first slot while the lid has a first orientation with respect to the base,
blocking, due to the shape and the dimensions of the first flap, of the first flap athwart the first slot by rotation of the lid from the first orientation to a second orientation with respect to the base, in which the lid covers, at least in part, the receiving zone.

The first flap used during this method has a bent portion forming an abutment for retaining the first flap athwart the first slot when the lid is in the second orientation with respect to the base, and/or the lid used during this method has a flat surface intended to rest against the base after engagement of the first flap in the first slot, and in which the axis of rotation of the first orientation toward the second orientation is perpendicular to the flat surface.

It can be provided that a second slot is formed in one among the base and the lid, and a second flap is formed in the other among the base and the lid, in which case the method also comprises the following step:
after engagement of the first flap in the first slot, then rotation of the lid from the first orientation to the second orientation with respect to the base, engagement of the second flap in the second slot, so as to block the lid in rotation with respect to the base in the second orientation.

### DESCRIPTION OF THE FIGURES

Other features, goals and advantages of the invention will be revealed by the description that follows, which is purely illustrative and not limiting, and which must be read with reference to the appended drawings in which:

FIG. 1 shows a top view of a base of the packaging for a suture according to a first embodiment of the invention.

FIG. 2 shows a top view of a lid of the packaging for a suture according to the first embodiment of the invention.

FIG. 3 is a detailed top view of the lid of FIG. 2 at a flap formed in it.

FIGS. 4 and 5 are respectively a top and bottom view of the base of FIG. 1 and of the lid of FIG. 2, during a first step of a method for locking the lid to the base.

FIG. 6 is a bottom view of the base of FIG. 1 and of the lid of FIG. 2, during a second step of the method for locking the lid to the base.

FIG. 7 is a partial top view of the base of FIG. 1 and of the lid of FIG. 2, forming together a closed package.

FIG. 8 is a top view of the base of FIG. 1 and of the lid of FIG. 2, forming together a closed package.

FIG. 9 is a top view of a packaging for a suture according to a second embodiment of the invention.

In all the figures, similar elements bear identical reference symbols.

### DETAILED DESCRIPTION OF THE INVENTION

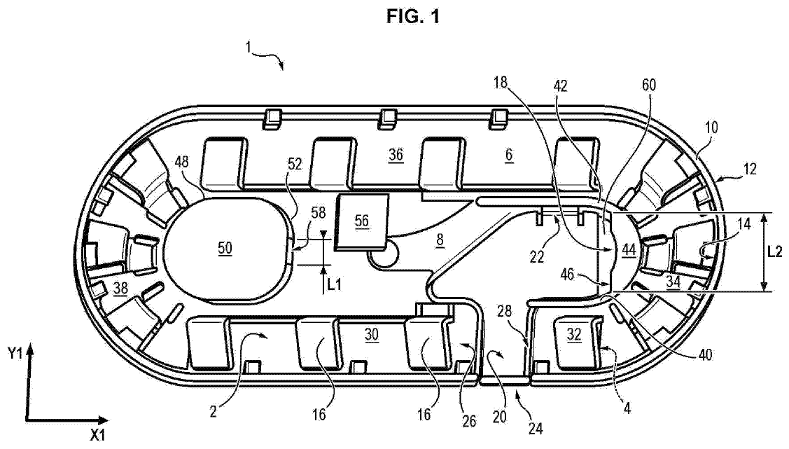

Referring to FIG. 1, a packaging for a suture comprises a base 1 forming a support for a suture.

The base 1 extend in a plane defined by two axes X1 and Y1.

The base 1 has a racetrack shape in this plane.

The base 1 comprises an upper side 2, visible in FIG. 1, and a lower side 4 opposite to the upper side 2.

The base 1 comprises, on the upper side 2, a receiving zone for receiving the suture.

The receiving zone comprises a channel 6 for retaining the suture in a wound position, and a parking zone 8 for at least one end of the suture wound in the channel, the parking zone 8 being surrounded by the retaining channel 6.

The retaining channel 6 is defined by an outer peripheral wall 10 and at least one inner wall 11, the two walls 10, 11 protruding from the upper side 2. In other words, the retaining channel 6 is defined between the walls 10, 11.

The peripheral wall 10 has an outer surface 12 forming a free outer edge of the base 1. This free edge 12 is graspable by an operator. The peripheral wall 10 also has an inner surface 14 opposite to the outer surface 12. The inner surface 14 delimits the retaining channel 6 on the outside.

The inner wall 11 comprises a plurality of wall elements surrounded by the outer wall 12; in other words, the wall 11 is discontinuous. As a variant, the wall 11 is continuous.

Retention fingers 16 extend as cantilevers from the wall 11 toward the wall 12 in a plane parallel to the plane (X1, Y1). A gap is left between one free end of each retaining finger 16 and the peripheral wall 10, so as to allow the insertion of a suture in the retaining channel via these gaps. However, each finger 16 overhangs the retaining channel 6 so as to prevent an untimely departure of a suture received in the retaining channel 6. A more detailed description of fingers of this type can be found in document WO2016066767.

The outer peripheral wall 10 has a greater height than the height of the inner wall 11.

The base 1 also has a through openings 18 between its two lower 4 and upper 2 opposite sides. This through opening 18 has a first part extending in the central parking zone 8. The first part is continued by a second part which extends in the peripheral zone so as to locally interrupt the retaining channel 6. The second part of the through opening 18 is also continued until the peripheral wall 10 so as to interrupt it locally. The through opening 18 thus forms a bay in the plane (X1, Y1) of the base 1, the bay being accessible from the free edge 12 of the base 1 formed by the peripheral wall 10.

The base 1 also comprises a movable shutter 20 between an open position for revealing the through opening 18 between its two sides 2 and 4, and a closed position for partially blocking the through opening 18. The movable shutter 20 has a connection 22 mounted pivotally to a part of the base 1 cohesive with the peripheral wall 10, and a free edge 24 opposite to the connection 22 and arranged to be aligned with the peripheral wall 12 when the movable shutter 20 is positioned in its closed position, as can be seen in FIG. 1.

Being interrupted locally by the through opening 18, the retaining channel 6 has two openings 26, 28 leading directly into this through opening (at the second part of the through opening 18 interrupting the retaining channel 6). The two openings 26, 28 are formed in two aligned rectilinear segments 30, 32 of the retaining channel 6.

The retaining channel 6 comprises the following successive segments: the rectilinear segment 32 terminated by the opening 28, a first curved segment 34 turning 180 degrees, a rectilinear segment 36 parallel to the segment 22, then another curved segment 38 turning 180 degrees, then finally the segment 30 terminated by the opening 26. The two curved segments 34 and 38 are for example symmetrical and situated on either side of the parking zone 8. The curved segments 34 and 38 have a C shape overall.

The two openings 26 and 28, separated by the through opening 18, face one another and are aligned so as to allow the suture to be wound in the retaining channel 6 in an ellipsoidal trajectory, free of sudden deviations likely to locally wear the suture at the moment of its dispensing and/or cause resistance during its dispensing.

The base 1 also includes a stiffening rib 40 protruding from the upper side 2 of the base 1.

The stiffening rib 40 extends between the through opening 18 and the retaining channel 6, at its curved segment 34. A rib 40 of this type increases the stiffness of the segment 34 despite the presence of the through opening 18, and thus allows facilitating the filling of a mold used for the manufacture of the base 1 support 1.

The stiffening rib 40 comprises a curved wall 42 with a C shape, which follows the retaining channel 6 and more precisely the inner wall 11 of the latter.

The stiffening rib 40 also comprises a wall 44 of which one substantially rectilinear edge 46 extend inside the C formed by the curved wall 42 of the stiffening rib 40. The base 1 also comprises a boss 48 protruding from the upper side 2 of the base 1, in the parking zone 8.

The boss 48 has an apex 50. The apex 50 is substantially flat, with a rectangular shape with rounded corners. The apex 50 extends in a plane parallel to the plane (X1, Y1).

The boss 50 also comprises a lateral wall 52 connecting the apex 50 to the base 1. The lateral wall 52 extends substantially perpendicular with respect to the body of the base 1 in the parking zone 8 and with respect to the apex 50.

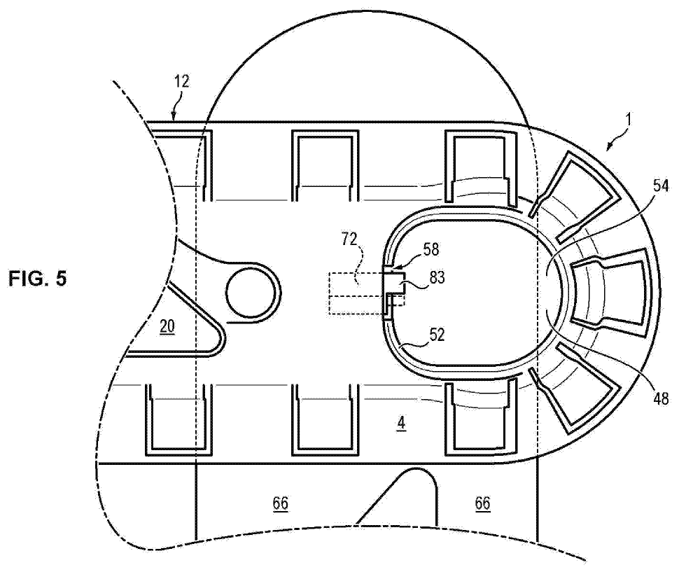

The boss 48 is hollow. It defines a cavity accessible from the lower side 4 of the base 1. The bottom of this cavity is formed by an inner surface 54 of the boss 48 (not visible in FIG. 1 but visible in FIG. 5). The boss 48 is a portion of the base 1 locally elevated with respect to the rest of the parking zone 8, this elevated portion being intended to serve as a support for a lid 62 of the packaging with will be described hereafter. This locally elevated portion also offers a grasping zone for a user. The user can in fact pinch this portion by means of a finger placed on the apex 50 and another finger placed on the inner surface 54.

Moreover the base 1 comprises a support 56 for one end of a suture. The support 56 is attached to the upper side 2, in the parking zone 8. The support 56 is localized between the boss 48 and the through opening 18. The support 56 is for example a foam into which a needle forming one end of a suture can be stuck and from which the needle can easily be removed. The support 56 is positioned in the parking zone 8 so that a needle stuck into this support 56 can partially overhang the through opening 18 (typically 1/3 of the needle). In this manner, a needle of this type stuck into the support 56 can be seized by means of an instrument introduced into the through opening 18 by the lower side 4, in the manner described in document WO2016066767.

Moreover, the base 1 has a first slot 58 and a second slot 60. As will be seen hereafter, these two slots 58, 60 have the function of locking the lid 62 of the packaging to the base 1.

By convention, the length of each slot 58, 60 is measured parallel to the axis Y1, their width is measured parallel to the axis X1, and their height is measured perpendicular to the plane (X1, Y1).

The two slots lead into the upper side 2, i.e. the side of the base 1 on which the receiving zone for the suture is formed. They also lead into the lower side 4 of the base 1.

More particularly, the two slots 58, 60 are formed in the parking zone 8 of the base 1, surrounded by the retaining channel 6.

The first slot 58 is formed in the lateral wall 52 of the boss 48, the first slot 58 is more precisely formed in a portion of the lateral wall of the boss 48 facing the through opening 18. The first slot 58 has a rectangular shape. Its length is denoted L1. Its height is a small dimension by comparison with its length L1.

The second slot 60 constitutes a part of the through opening 18.

The second slot 60 is delimited by the movable shutter 20 and the stiffening rib 40. More precisely, the second slot 60 is formed between the substantially rectilinear edge 46 of the wall 44 of the stiffening rib 40, and an edge of the movable shutter 20 substantially parallel to the edge 46.

Preferably, the wall 44 has a small rounded recess, so that the substantially rectilinear edge 46 has a curved central portion.

The first slot 58 has a substantially rectangular shape. It has a length L1 and a width that is a small dimension in comparison to its length L1.

The two slots 58, 60 are substantially parallel with the axis Y1.

The base 1, formed in one piece, is manufactured by molding. The base 1 is for example made of plastic.

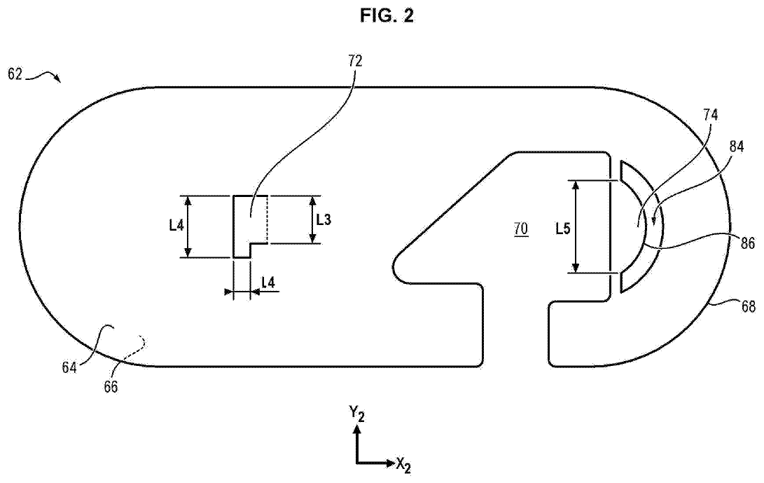

With reference to FIG. 2, the packaging comprises moreover a lid 62.

The lid 62 is a sheet made of a flexible material, typically paper or cardboard, possibly plasticized. The sheet extends in a plane defined by two axes X2 and Y2.

The lid 62 is intended to cover at least partially the suture receiving zone formed on the upper side 2 of the base 1.

The lid 62 provides in particular the following functions:
contributes to protecting a suture received in the receiving zone of the base, and in particular,
provides humidity to a suture contained in the packaging, during a method for sterilizing this suture using ethylene oxide;
in the case of resorbable sutures, guarantees a low level of humidity in the resorbable suture contained in the packaging. In fact, the humidity will attach itself first and foremost to the cardboard of the lid rather than to the suture.
serves as an information medium.

The lid 62 has an upper side 64 and a lower side 66 opposite to the upper side 64. The lower side 66 is intended to be pressed against the base 1, and the upper side 64 is intended to form an outer surface of the packaging formed by the assembly of the base 1 and the lid 62. Information relating to a suture intended to be received in the packaging is typically written on the upper side 66.

The lid 62 has an outer peripheral edge 68 substantially complementary to the inner surface 14 of the outer peripheral wall 10 of the base 1. Thus, the lid 62 can be placed on the upper side 2 of the base 1 so as to be surrounded by the outer peripheral wall 10, without leaving any gap between the outer peripheral edge 68 of the lid 62 and the peripheral wall 10 of the base 1. In such a position of the lid 66 with respect to the base 1, called the closed position, the orientation of the lid 62 relative to the base 1 is such that the axis X2 is parallel to the axis X1, the axis Y2 is parallel to the axis Y1, and the lower surface 66 of the lid 62 rests against the apex 50 of the boss 48 and against the retaining fingers 16.

Moreover, the lid 62 has a through opening 70 having the same shape as the through opening 18 formed in the base 1.

The through opening 70 is arranged so as to be superimposed at least in part with the through opening 18 of the base 1, when the lid 62 is positioned on the base 1 in the closed position.

The lid 62 comprises a first flap 72 as well as a second flap 74. The geometry, the location and the dimensions of the flaps 72, 74 are discussed hereafter. By convention, the length of a part of one of the flaps is measured parallel to the axis Y2 and its width is measured parallel to the axis X2.

The first flap 72 is intended to be engaged athwart the first slot 58 of the base 1.

The first flap 72 is formed by simply cutting out the sheet 62.

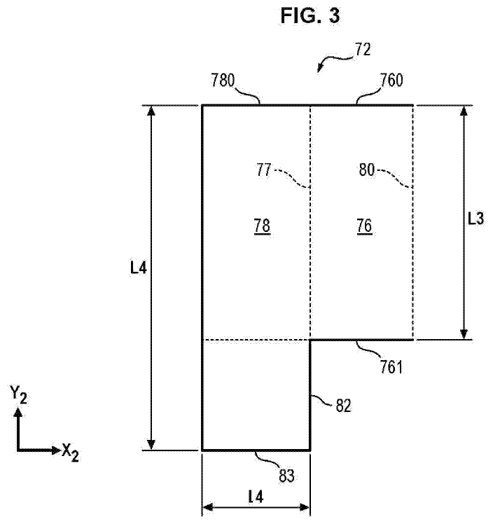

Referring to FIG. 3, the first flap 72 has the shape of an L.

The first flap 72 comprises and intermediate portion 76 and a free end portion 78.

The intermediate portion 76 is intended to extend athwart the first slot 58. The intermediate portion 76 is connected to the rest of the sheet of the lid 62 by a rectilinear fold line 80. The fold line 80 is parallel to the axis Y2. The fold line 80 facilitates pivoting of the first flap 72.

Alternatively, the line 80 is not a fold line but simply a demarcation line between the second flap 74 and the rest of the lid 62, so as to allow the flap to return naturally into a position substantially parallel to the rest of the lid 62, and also to make the flap more robust.

The intermediate portion 76 has a length L3 less than or equal to the length L1 of the first slot 58.

The free end portion 78 continues the intermediate portion 76 opposite to the fold line 80; a bend 82 of the first flap 72 is formed in the portion 78 (this bend 82 can also be considered one point of the L).

The free end portion 78 has a length L4 greater than the length L1 of the first slot 58. As will be seen hereafter, this allows preventing the first flap 72 from re-departing from the first slot 58, once the flap 72 is engaged in the slot 58 so that the free end portion 78 of the flap 72 leads out of the slot 58 on the lower side 4 of the base 1, this when the lid 62 is in its closed position.

The free end portion 78 has a lateral edge 83 perpendicular to the fold line 80.

More precisely, the two intermediate 76 and free end 78 portions are separated by a line 77 parallel to the fold line 80. Moreover, they are delimited by lateral edges 760, 761 for the intermediate portion 76 and 780, 83 for the free end portion 78 which extend perpendicular to the fold line 80. The two lateral edges 760 and 780 are aligned.

On the other hand, the free end portion 78 being longer than the intermediate portion 76, considered parallel to the axis Y2, the edges 761 and 83, mutually parallel, are not aligned. The edge 83 of the free end 78 is situated beyond the free edge 761 of the intermediate portion 76. The edges 761 and 83 are separated by the length of the bend 82.

The free end portion 78 has a length L4 less than the length L1 of the first slot. This allows insertion of the free end portion 78 into the first slot, when the lid 62 is oriented at 90 degrees with respect to the base 1 (i.e. when the axes X1 and X2 are perpendicular and when the axes Y1 and Y2 are also perpendicular, the two planes X1, Y1 and X2, Y2 remaining mutually parallel).

The two portions 76, 78 of the first flap 72 have for example a rectangular shape.

Returning to FIG. 2, the second flap 74 is intended to be engaged in the second slot 60.

The second flap 74 has a length L5 less than the length L2 of the second slot 60.

The second flap 74 has moreover a convex curved free edge 86, shaped for example like a portion of a circle.

The second flap 74 is formed in the sheet of the lid 62 after cutting out a C-shaped opening 84 in it. The curved free edge 86 of the second flap 74 is a part of the edge of this C-shaped opening.

Alternatively, the second flap is formed by simply cutting out the lid, for example in a C shape, without forming an opening 84.

The second flap 74 is connected to the base by a second line extending parallel to the fold line 80. The second line, however, is not a fold line; it is simply a demarcation line between the second flap 74 and the rest of the lid 62. It is nevertheless possible to provide that the second line is a second fold line parallel to the fold line 80.

The distance between the two lines, measured parallel to the axis X2 is substantially equal to the distance separating the complementary slots which receive the flaps 58, 60. Thus the flaps 72 and 74 extend on the outside of the slots after introduction.

The two flaps 72, 74 extend in two opposite directions. In this manner, when the lid 62 is in its closed position, an accidental departure of the second flap 74 out from the second slot 60 by flexure of the lid is made less easy.

The two slots and the two complementary flaps are disposed in the vicinity of the longitudinal ends of the base and of the lid with an elongated, racetrack shape.

The packaging comprises means for locking the lid 62 to the base 1.

These locking means comprise the slots 58, 60 formed in the base and the flaps 72, 74 formed in the lid 62. This locking function is more amply described hereafter.

Method for Locking the Lid to the Base

The lid 62 is locked to the base 1, so as to form a closed package for a suture, in the following manner.

A suture was previously wound into the retaining channel 6. At least one end of the suture comprising a needle (for example two opposite ends of the same suture) escapes from the retaining channel 6 through one of the openings 24 and 26. The needle of this suture is stuck into the support arranged in the parking zone 8.

The lower surface 66 of the sheet of the lid 62 is positioned against the upper side 2 of the base 1. The lid 62 oriented at 90 degrees with respect to the lid 62, (i.e. when the axis X1 and X2 are perpendicular and when the axes Y1 and Y2 are also perpendicular). In this orientation, the lateral edge 83 of the free end portion 78 of the first flap 72 is engaged in the first slot 58 of the base 1 by the upper side 2, by translation of the lid 62 relative to the base 1.

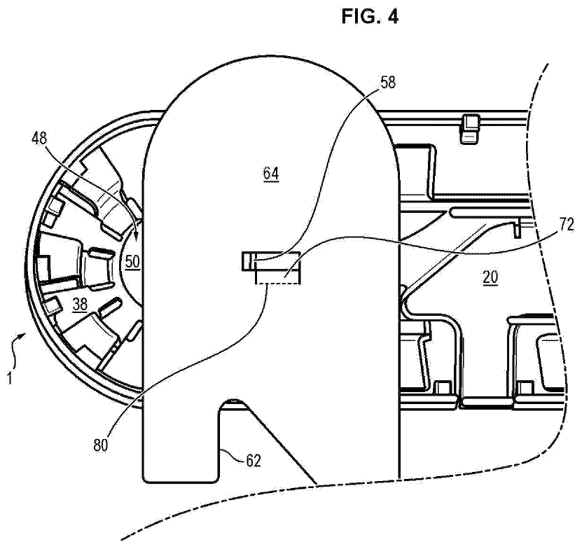

To penetrate into the first slot 58, the first flap 72 is slightly inclined with respect to the rest of the sheet forming the lid 62, by folding along the fold line 80, as can be seen in FIG. 4. Nevertheless, as the first slot 58 is formed in the lateral wall 52 of the boss 48, this engagement is carried out without having the first flap 72 heavily loaded in flexure. The engagement of the first flap 72 in the first slot 58 is thereby facilitated.

The lateral edge 83 of the free end portion 78 of the first flap 72 leaves the slot 58 by the lower side of the base 1, thus finds itself in the inner cavity defined by the boss 48, on the lower side 4 of the base 1, as shown in FIG. 5.

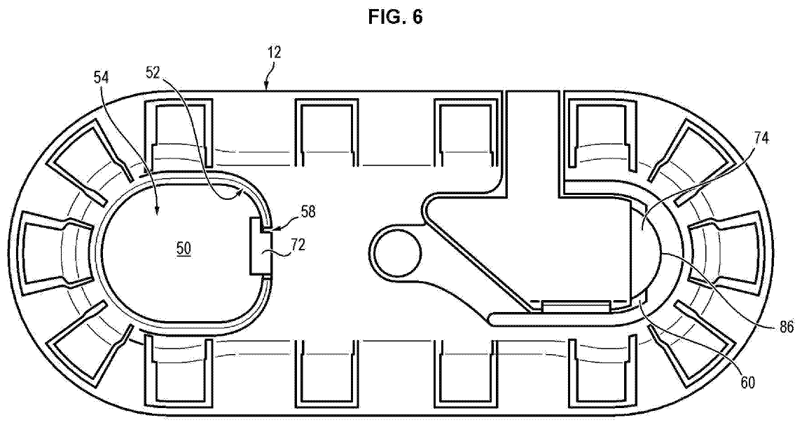

The lid 62 is then rotated around an axis perpendicular to the planes (X1, Y1) and (X2, Y2), over an angle of approximately 90 degrees, so that the lid 62 reaches the closed position described previously. During this rotation, the free end portion 78 of the first flap 72 is engaged entirely in the first slot 58 of the base 1 by the upper side 2, then entirely leaves the slot 58 of the lower side 4 of the base. The bend 82 formed in this free end portion is aligned with the inner surface of the lateral wall of the boss 48 in the vicinity of the first slot 58, thus forming an abutment which prevents departure of the first flap 72 out of the first slot 58, as can be seen in FIG. 6.

Moreover, during this rotation, the second flap 74 is brought closer to the second slot 60 until the curved edge of the second flap 74 enters into contact with the wall 46 of the rib defining one edge of the second slot 60. The second flap 74 is engaged in the second slot 60 by slightly folding the second flap 74 with respect to the rest of the sheet of the lid 62. The second flap 74 is engaged in the second slot 60 by slightly flexing the second flap 74 with respect to the rest of the sheet of the lid 62. This flexure can be initiated either during the rotation to the closed position, or once the lid has attained the closed position. The fact that the second flap 74 has a curved shape allows facilitating sliding the second flap 74 below the wall 44 defining the edge 46 of the second slot 60.

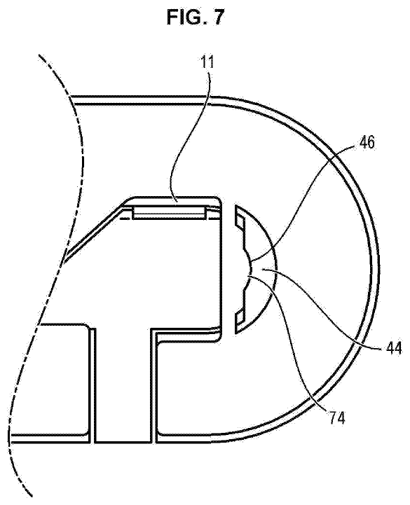

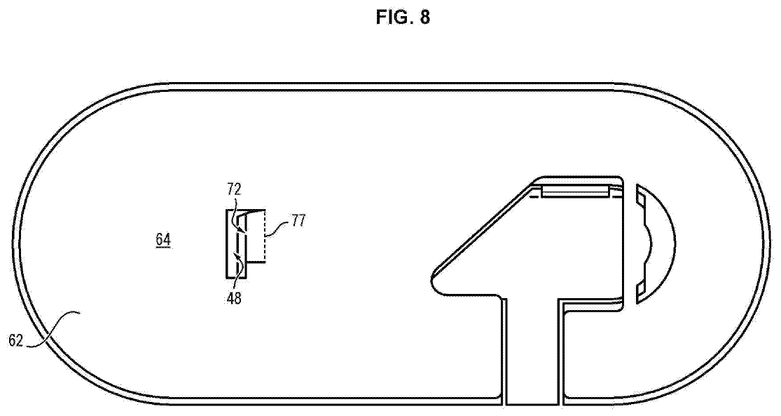

FIG. 7 shows the second flap 74 engaged in the second slot 60 while the lid 62 is in the closed position, and FIG. 8 is a more general view of the packaging when the lid 62 is in the closed position.

Once the second flap 74 has been engaged in the second slot 60, the lid is blocked in rotation, which has the effect of more solidly locking the lid 62 to the base 1.

In the closed position, the lid 62 covers at least in part the receiving zone of the suture, so as to protect the latter.

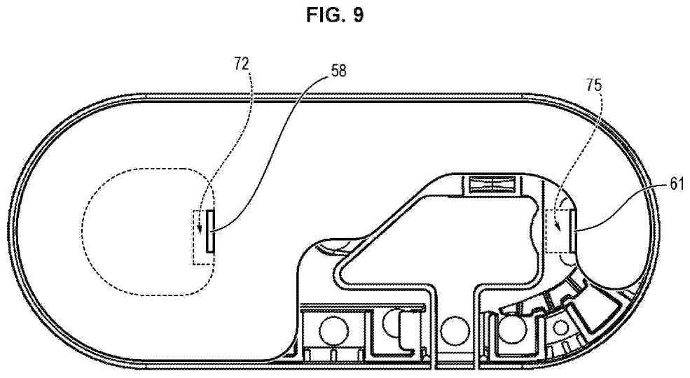

Shown in FIG. 9 is packaging for a suture according to a second embodiment, in which is found most of the features of the first embodiment, in particular the first slot 58 and the first flap 72. This packaging differs, however, from the packaging according to the first embodiment shown in FIGS. 1 to 8 in that it comprises a second flap 75 formed in its lid, and a second slot 61 formed in its base, which are oriented differently from the flap 74 and the slot 60. In fact, the second flap 75 of the packaging according to the second embodiment is oriented in the same direction as the first flap 72, and not in an opposite direction. Moreover, the second slot 61 is arranged to allow a double insertion of the two flaps 72, 75 into the two slots 58, 61, as was already the case in the packaging according to the first embodiment. In other words, the packaging according to this second embodiment comprises two flaps 72, 75 extending so as to be able to be inserted respectively into the two slots 58, 61 formed in the base, in the same direction. Such a disposition of the flaps has the advantage of avoiding even more effectively an accidental departure of the second flap 75 out of the second slot 61, compared to the first embodiment. In fact, the first flap 74 is prevented from departing from the first slot 58 due to its bent shape.

The invention is not limited to the embodiments shown in the figures. In particular, although an L shape is particularly advantageous for the first flap due to its nature which is easy to manufacture and to engage in the first slot 58, the first flap can have other shapes adapted to be blocked athwart the first slot 58 following rotation of the lid with respect to the base.

It can be provided that the first flap has a shape adapted so that its blocking can be implemented after a rotation by an angle less than or greater than 90 degrees with respect to the lid.

1. Packaging for a suture, the packaging comprising:
a base (1) comprising a receiving zone (6, 8) for receiving a suture,
a lid (62),
means for locking the lid (62) to the base (1), said means comprising a first slot (58) formed in one among the base (1) and the lid (62), and a first flap (72) formed in the other among the base (1) and the lid (62),
wherein the first flap (72) has a shape and dimensions adapted to:
be engaged through the first slot (58) when the lid (62) has a first orientation with respect to the base (1), then
be blocked athwart the first slot (58) by rotation of the lid (62) from the first orientation to a second orientation of the lid with respect to the base wherein the lid covers at least in part the suture receiving zone (6, 8),
wherein the first flap (72) has a bent portion (82) forming an abutment for retaining the first flap (72) athwart the first slot (58) when the lid (62) is in the second orientation with respect to the base (1), and/or wherein the lid has a flat surface (66) intended to rest against the base (1) after engagement of the first flap (72) in the first slot (58), and the axis of rotation of the first orientation toward the second orientation is perpendicular to the flat surface (66).

2. Packaging according to claim 1, wherein the first flap (72) comprises a portion (78) having:
a dimension (14), measured parallel to the first slot (58) when the lid is in the first orientation with respect to the base, which is less than a length (L1) of the first slot (58), and
a dimension (L4), measured parallel to the first slot (58) when the lid (62) is in the second orientation with respect to the base, which is greater than the length (L1) of the first slot (58).

3. Packaging according to claim 1, wherein the first flap (72) has an L shape.

4. Packaging according to claim 1, wherein the first slot (58) is formed in the base (1), and the first flap (72) is formed in the lid (62).

5. Packaging according to claim 1, wherein the lid (62) comprises a sheet, the first flap (72) being formed by cutting out the sheet.

6. Packaging according to claim 1, comprising a boss (48) protruding from the base (1), the boss (48) having an apex (50) able to serve as a support for the lid (62), and a lateral wall (52) connecting the apex (50) to the base (1), the first slot being formed in the lateral wall (52) of the boss (48).

7. Packaging according to claim 1, wherein the locking means further comprise:
a second slot (60, 61) formed in one among the base (1) and the lid (62),
a second flap (74, 75) formed in the other among the base (1) and the lid (62), the second flap (74, 75) having a shape and dimensions adapted to be engaged in the second slot (60, 61) after engagement of the first flap (72) through the first slot, then rotation of the lid (62) from the first orientation to the second orientation with respect to the base (1), so as to block the lid (62) in rotation with respect to the base (1) in the second orientation.

8. Packaging according to claim 7, wherein the second slot (60, 61) is formed in the base (1) and the second flap (74) is formed in the lid (62).

9. Packaging according to claim 7, wherein the first and second flaps (72, 75) extend so as to be inserted respectively in the first and second slots (58, 61) in a same direction.

10. Packaging according to claim 1, wherein the base (1) comprises a peripheral wall (10) having an inner surface (14), the lid (62) has an outer edge (68) with a shape complementary to the inner surface (14) of the peripheral wall (10), the peripheral wall (10) being arranged so as to surround the lid (62) when the lid has the second orientation after engagement of the first flap (72) through the first slot (58).

11. Packaging according to claim 1, wherein the base (1) comprises a channel (6) for retaining a suture in a wound position, and a parking zone (8) for one end of the suture, the parking zone (8) being surrounded by the retaining channel (6), and wherein the first slot (58) or the first flap (72) is formed in the parking zone (8).

12. Packaging according to claim 1, further comprising a suture received in the receiving zone of the base (1).

13. A method for locking a lid (62) of a packaging for a suture to a base (1) of the packaging, wherein the base (1) comprises a receiving zone for receiving the suture, and wherein a first slot (58) is formed in one among the base (1) and the lid (62) and a first flap (72) is formed in the other among the base (1) and the lid (62), wherein the method comprises:
engaging the first flap (72) through the first slot while the lid (62) has a first orientation with respect to the base (1),
blocking, due to the shape and the dimensions of the first flap (72), of the first flap (72) athwart the first slot (58) by rotation of the lid (62) from the first orientation to a second orientation with respect to the base (1), in which the lid (62) covers at least in part the receiving zone (6, 8),
wherein the first flap (72) has a bent portion (82) forming an abutment for retaining the first flap (72) athwart the first slot (58) when the lid (62) is in the second orientation with respect to the base (1), and/or wherein the lid has a flat surface (66) intended to rest against the base (1) after engagement of the first flap (72) into the first slot (58), and the axis of rotation of the first orientation toward the second orientation is perpendicular to the flat surface (66).

14. The method according to claim 13, wherein the second slot (60, 61) is formed in one among the base (1) and the lid (62) and a second flap (74, 75) is formed in the other among the base (1) and the lid (62), and also comprising the following step:
after engagement of the first flap (72) in the first slot (58), then rotation of the lid (62) from the first orientation to the second orientation with respect to the base (1), engagement of the second flap (74, 75) in the second slot (60, 61) so as to block the lid (62) in rotation with respect to the base (1) in the second orientation.
